(19) United States
(12) Patent Application Publication
(10) Pub. No.: US 2016/0374749 A1
(43) Pub. Date: Dec. 29, 2016

(54) STRUCTURAL ARRANGEMENT FOR A SURGICAL INSTRUMENT HANDLE

(71) Applicant: IMBROS ADMINISTRAÇÃO E PARTICIPAÇÕES LTDA, Porto Alegre (BR)

(72) Inventor: Alessandro TERZARIOL, Chiusa (IT)

(21) Appl. No.: 15/111,121

(22) PCT Filed: Oct. 20, 2015
(86) PCT No.: PCT/BR2015/050186

§ 371 Date: Sep. 16, 2016

(30) Foreign Application Priority Data
Oct. 20, 2014 (BR) 202014026180-5

(51) Int. Cl. A61B 18/14

(52) U.S. Cl. CPC A61B 18/1445; A61B 2017/2925

(57) ABSTRACT

A structural arrangement for a surgical instrument handle has a vertical structure (10) having in the upper portion thereof a connection region (11) for connecting to a horizontal structure (20) which can pivot relative to the horizontal axis of the region (11), and an arm (30) which projects downwards from the vertical structure (10) and has an oval structure (32) which can be fitted into the inner cavity of the arm (30), allowing the thumb to move when the handle is used, thus providing an instrument that is firm, soft and easy to handle.

### CROSS-REFERENCE TO RELATED APPLICATIONS

This application is a national phase application of PCT/BR2015/050186 filed Oct. 20, 2015, which claims priority to Brazilian App. No. 202014026180-5 filed Oct. 20, 2014, the disclosures of which are hereby incorporated by reference.

### BACKGROUND AND SUMMARY OF THE DISCLOSURE

The present disclosure describes a structural arrangement for surgical instrument handle. More specifically, it comprises a vertical structure having in the upper portion thereof a connection region with a rotating horizontal structure provided with a quick engagement system and arm downwardly projected from vertical structure that has an oval rotating hollow structure, resulting in a firm, smooth and easy instrument to handle.

The handles allow the manipulation of instruments, allowing fine control of the positioning of the tweezers and dissectors, thereby facilitating handling.

Currently, the handles have the most varied forms and models, and the main feature is the mechanical functionality to perform the necessary movements of the tweezers for surgical procedures, wherein these products do not give priority to the ergonomic issue of handles.

The state of the art discloses handles provided with arms that have in their lower portion spacings for positioning of the fingers, where the movement of approach and separation from the thumb and index/middle fingers corresponds directly to the opening and closing of functional stems of the instrument. In addition, it describes a threaded system arranged with the nozzle, which allows the attachment of surgical instruments.

However, the consumer market demands greater speed and efficiency in procedures, so the assembly of products with threaded nozzle systems no longer meets the current market demand, requiring a product provided for easy docking and handling system.

Thus, the object of this disclosure is a vertical structure having in the upper portion thereof a connection region with a rotating horizontal structure provided with a quick engagement system and an arm downwardly projected from vertical structure that has an oval rotating hollow structure, resulting in a firm, smooth and easy instrument to handle.

These features promote simplicity and greater speed and ease of product handling during the procedure, when compared with products of prior art.

It is a characteristic of the disclosure that a structural arrangement for a surgical instrument handle provides an engagement system for assembling and disassembling the product quickly and easily.

It is a characteristic of the disclosure that a structural arrangement for a surgical instrument handle provides a handle system with ergonomic design that reduces product handling difficulties during the procedure, enabling the perfect fit in the hand.

It is a characteristic of the disclosure that a structural arrangement for a surgical instrument handle provides an engagement system of the rotating thumb that facilitates the movements during use.

### DESCRIPTION OF THE DRAWINGS

In order to better describe the technical characteristics of the structural arrangement for surgical instrument handle, the following figures are shown below:

FIG. 1 shows the perspective view of the surgical instrument handle.

FIG. 2 shows the lateral view of the surgical instrument handle.

FIG. 3 shows the frontal view of the surgical instrument handle.

FIG. 4 shows the upper view of the surgical instrument handle.

FIG. 5 shows the exploded view of the horizontal structure, the FIG. 5A shows detailing of the engagement of nozzle with the horizontal structure and the FIG. 5B shows the perspective view of the horizontal structure assembled with the nozzle.

FIG. 6 shows the perspective view of the handle describing the movement direction of the oval structure and

FIG. 6A shows the section detailing of the fastening of oval rotating structure along the handle arm.

FIG. 7 shows the exploded view of the surgical instrument handle.

FIG. 8 shows the lateral view of the structural variation of surgical instrument handle.

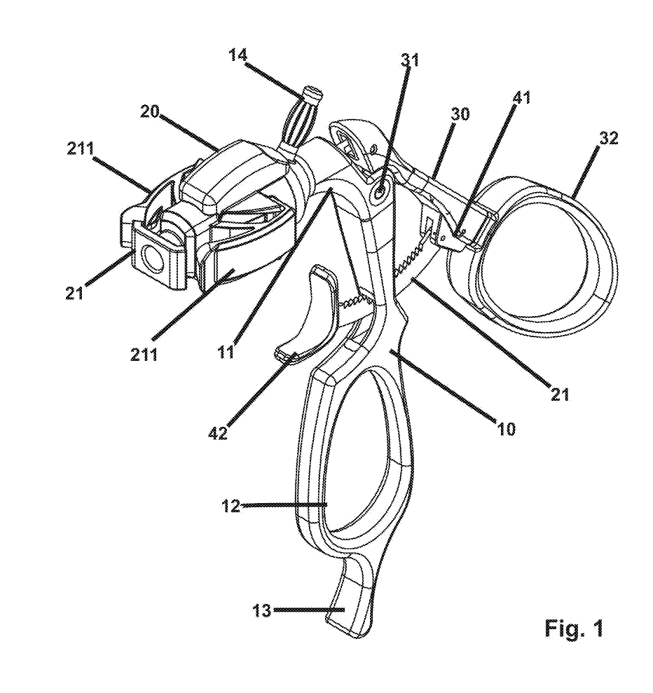

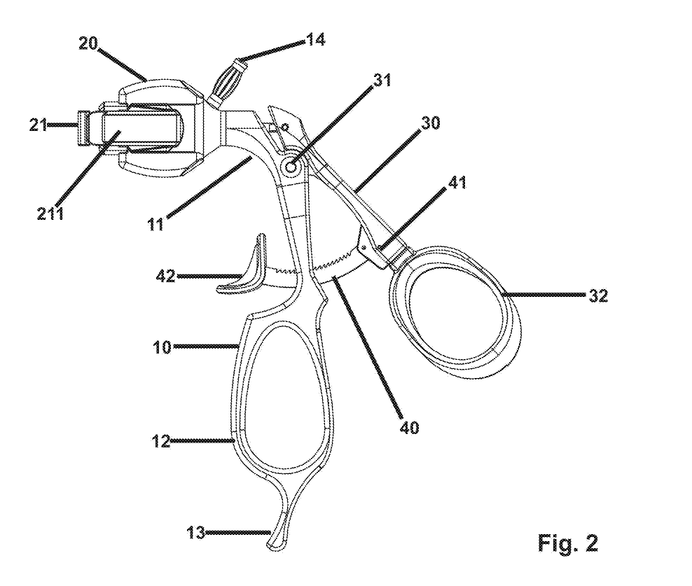

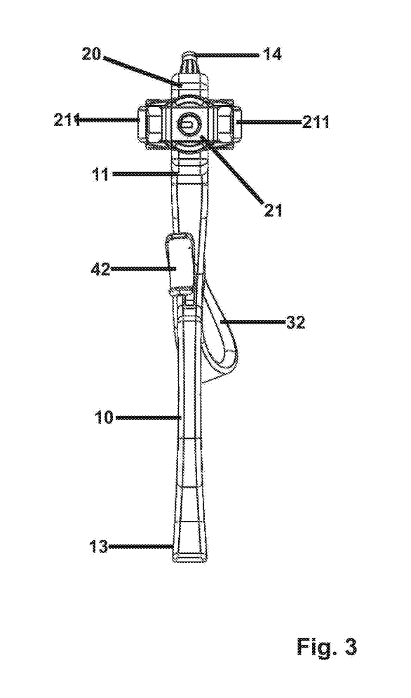

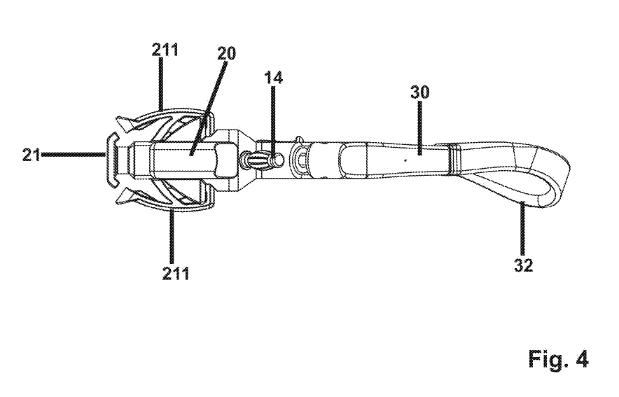

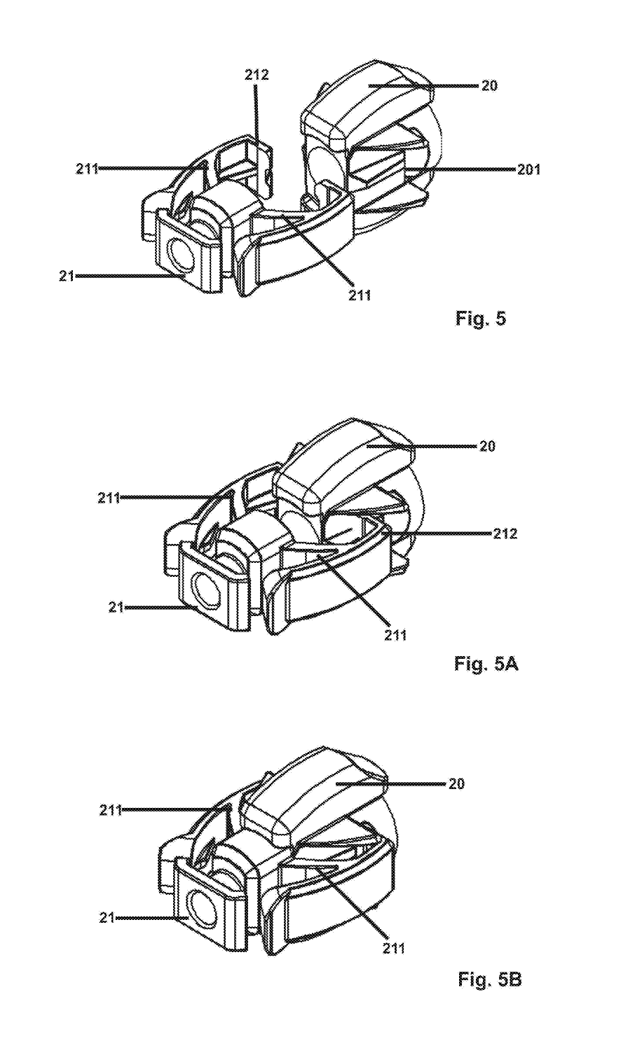

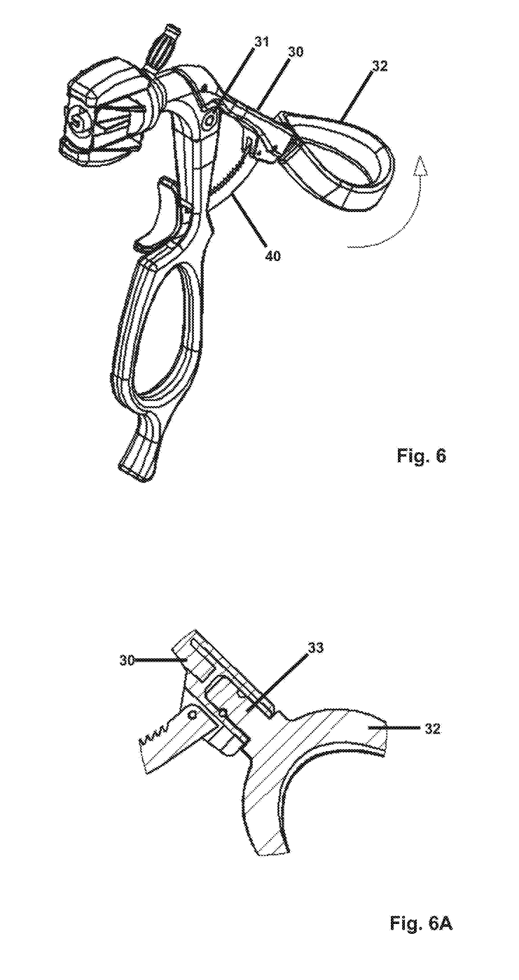

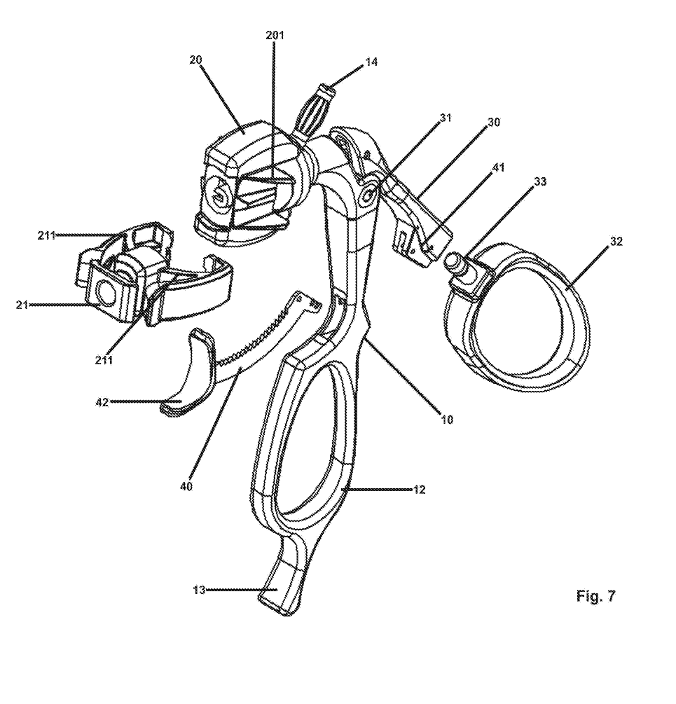

### DETAILED DESCRIPTION OF THE DISCLOSURE

The structural arrangement for surgical instrument handle, object of the present disclosure comprises a vertical structure (10) having in the upper portion thereof a connection region (11) along a rotating horizontal structure (20) in relation to the horizontal axis of the region (11), and an arm (30) downwardly projected from the vertical structure (10).

The vertical structure (10), having in the upper portion thereof a connection region (11) along a horizontal structure (20), describes from the end opposite to horizontal structure (20) the inclination which results in a hollow oval structure (12) arranged in the lower region of the structure (10), said oval structure (12) provided with a curved tab (13) of support and sustaining at the time of handling.

The rotating horizontal structure (20) in relation to the horizontal axis of the region (11) comprises in the end opposite to the connection region (31) the fastening nozzle (21) of surgical instruments, said fastening nozzle (21) comprising lateral projections (211) which allow the engagement and disengagement of surgical instruments. Furthermore, it comprises in the upper portion the connector (14) near the connection region (11).

The engagement system is based on a rotating ring fixed in the horizontal axis of the region (11) and has a sense of free rotation perpendicular to this horizontal axis. The engagement system of the nozzle (21) in the rotating horizontal structure (20) occurs by bending of the stems from both sides (211) and locks through the outermost points (212) in appropriate points (201) of the rotating structure.

The connector (14) has the purpose to perform the electric cable connection on the handle and transmit electrical power from electrocautery to the active part of the instrument.

The arm (30) downwardly projected from the vertical structure (10) is fixed along the structure (10) through an axis (31) arranged in the upper portion of the structure (10), said arm (30) having the inclination which results in a hollow oval structure (32) arranged in the lower region of the arm (30). Said hollow oval structure (32) comprises rotating region around the longitudinal axis of the arm (30), facilitating the movements while handling procedure.

Due to the circular connection of the hollow oval structure (32) aligned longitudinally to the arm (30), the hollow oval structure (32) can rotate perpendicular to the arm axis (30) due to pin (33) derived from oval structure (32) that fits the inner cavity of the arm (30), enabling the mobility of the thumb during use of the handle.

In the medial portion of the structure (10) and arm (30), it is described a tab with serration (40) running through the middle region of the vertical structures (10) and arm (30), said tab with serration (40) comprises in one of its ends of the fastening point (41) along the arm (30) and in its posterior end of the curvilinear tab (42) arranged along the vertical structure (10).

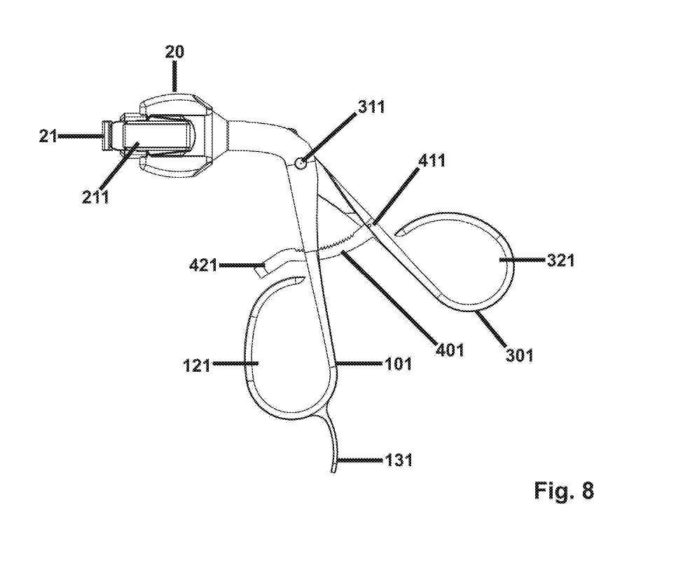

The structural arrangement for surgical instrument handle shows a structural variation in the vertical structure and arm, according to FIG. 8, which comprises a simplification of parts, describing an arm (301) downwardly projected from vertical structure (101) fixed along the structure (101) through an axis (311) arranged in the upper portion of the structure (101), said arm (301) having inclination which results in a hollow oval structure (321) arranged in the lower region of the arm (301). In addition, it shows a structural variation in the hollow oval structure (121) arranged in the lower region of the structure (101) provided with a curved tab (131) and a tab with serration (401) running through the middle region of the vertical structures (101) and arm (301), said tab with serration (401) comprises in one of its ends of the fastening point (411) along the arm (301) and in its posterior end of the curvilinear tab (421) arranged along the vertical structure (10).

Optionally, the structural arrangement for surgical instrument handle may be made of carbon fiber or other steel models, according to the need and specifications of use and handling of the handle.

1. STRUCTURAL ARRANGEMENT FOR SURGICAL INSTRUMENT HANDLE comprising a vertical structure (10) having in a upper portion thereof the connection region (11) and the connector (14), in the lower portion has an oval structure (12) and curved tab (13), said connection structure (11) shows, in its end, a rotating horizontal structure (20) in relation to the horizontal axis of the region (11), said horizontal structure (20) describing engagement points (201) of fastening of the nozzle (21) through flexing of the stems from both sides (211) and locking (212) of engagement to the point (201); the arm (30) downwardly projected from the vertical structure (10) is fixed along the structure (10) through and axis (31) arranged in the upper portion of the structure (10), said arm (30) comprises an inclination resulting in a hollow oval structure (32) arranged in the lower region of the arm (30) that promotes the rotation perpendicular to the arm axis (30) due to pin (33) derived from oval structure (32); in the medial portion of the structure (10) and arm (30), it is described a tab with serration (40) running through the middle region of the vertical structures (10) and arm (30), said tab with serration (40) comprises in one of its ends of the fastening point (41) along the arm (30) and in its posterior end of the curvilinear tab (42) arranged along the vertical structure (10).

2. STRUCTURAL ARRANGEMENT FOR SURGICAL INSTRUMENT HANDLE according to claim 1, comprising a structural variant comprising an arm (301) downwardly projected from vertical structure (101) fixed along the structure (101) through an axis (311) arranged in the upper portion of the structure (101), said arm (301) comprising an inclination resulting in a hollow oval structure (321) arranged in the lower region of the arm (301), said structure (101) provided with an inferior portion of a hollow oval structure (121) comprising a curved tab (131); in the medial portion, it comprises a tab with serration (401) running through the middle region of the vertical structures (101) and arm (301), said tab with serration (401) comprising in one of its ends of the fastening point (411) along the arm (301) and in its posterior end of the curvilinear tab (421) arranged along the vertical structure (10).

3. STRUCTURAL ARRANGEMENT FOR SURGICAL INSTRUMENT HANDLE according to claim 1, wherein optionally the handle is made of carbon fiber or other steel models, according to the need and specifications of use and handling of the handle.
